(19) United States
(12) Patent Application Publication
(10) Pub. No.: US 2011/0009630 A1
(43) Pub. Date: Jan. 13, 2011

(54) PROCESS FOR THE PREPARATION OF MELAMINE

(76) Inventor: Tjay Tjien Tjioe, Sinard (NL)

Correspondence Address: NIXON & VANDERHYE, PC, 901 NORTH GLEBE ROAD, 11TH FLOOR, ARLINGTON, VA, 22203, US

(21) Appl. No.: 12/809,303

(22) PCT Filed: Nov. 27, 2008
(86) PCT No.: PCT/EP08/10073

§ 371 Date: Oct. 1, 2010

(30) Foreign Application Priority Data
Dec. 20, 2007 (EP) 07024777.0

(51) Int. Cl. C07D 251/60

(52) U.S. Cl. 544/201

(57) ABSTRACT

The present invention relates to a non-catalytic process for the preparation of melamine comprising: a) reacting molten urea in a reaction section at a pressure of 4.5 to 15 MPa to produce a reaction mixture containing molten melamine and reaction off-gases; b) vaporizing the molten melamine in said reaction mixture in a vaporization section to produce a gaseous mixture comprising the vaporized melamine and the reaction off-gases at a pressure of 4.5 to 15 MPa; c) quenching the gaseous mixture obtained from step b) by contact with an aqueous ammonium carbamate solution; and d) isolating melamine; and to a combined process for the preparation of urea and melamine, wherein melamine is prepared in accordance to the above method wherein at least part of the concentrated aqueous ammonium carbamate solution is transferred to an urea plant as at least part of the feed stock, urea is prepared utilizing said concentrated aqueous carbamate solution and urea obtained from said preparation step is supplied in molten form to the reacting step a).

The present invention relates to a non-catalytic process for the preparation of melamine. According to another embodiment the present invention relates to a combined process for making melamine and urea.

### BACKGROUND OF THE INVENTION

It is well known that melamine can be prepared from urea at elevated temperature according to the following reaction equation:

6H₂N—CO—NH₂→C₃N₃(NH₂)₃+6NH₃+3CO₂

The reaction is strongly endothermic. The heat requirement is 649 kJ per mole melamine when the heating of the urea from 135° C. (melting point of urea) to the reaction temperature is included.

Users require a very high purity of melamine; 99.8% and 99.9% are typical degrees of purity in product specifications. For this reason its production processes often include a complicated purification section involving a large quantity of apparatus.

There are two basic types of melamine production processes using urea as the raw material, namely, catalytic low-pressure processes and high-pressure processes in which no catalyst is used. In the former, the reactor pressure is approx. 1 MPa or lower, in the latter reactor pressure is usually higher than 8 MPa (Ullmann's Encyclopedia of Industrial Chemistry, 5th edition. Vol. A 16, p. 174-179).

In typical low-pressure processes, a fluid-bed reactor is used in which a catalyst is fluidized with gaseous ammonia or with a mixture of ammonia and carbon dioxide. The melamine emerges in gaseous state from the reactor. The fact that corrosion is less than in high-pressure processes is regarded as one of the advantages of low-pressure processes.

In typical high-pressure processes the reaction takes placed in a liquid phase. In this case the reactor is full of molten melamine mixed to some degree with molten raw material, i.e. urea, and intermediate reaction products. Also present in the mixture there are gas bubbles consisting of ammonia and carbon dioxide and a small amount of gaseous melamine. The required high amount of reaction heat is usually generated by intra-reactor heating elements, in which the heat is generated by means of electricity or, for example, a circulating hot salt melt.

Smaller apparatus size is deemed to be one of the advantages of high-pressure processes over low-pressure processes. A reaction taking place in a liquid phase clearly requires less space. Furthermore, the process apparatus in which gas is treated remains moderate-sized owing to the high pressure. Another advantage is the high pressure of the obtained product gas, a mixture of ammonia and carbon dioxide. This gas is often used for the preparation of urea and, being pressurized, it is better suited for this purpose as such.

The Montedison process is a typical high-pressure melamine production process (Ullmann's Encyclopedia of Industrial Chemistry, 5th edition. Vol. A 16, p. 177). As in other melamine processes, urea melt and hot ammonia are introduced into a reactor. The reactor conditions are 7 MPa and 370° C. From the reactor the mixture of melamine melt and product gases is directed to a quencher, into which water containing ammonia and carbon dioxide is also introduced. The temperature of the quencher is 160° C. and its pressure is 2.5 MPa. From this quencher the reactor offgases are fed for further use, for example for the production of urea or fertilizers. The melamine is recovered from the resulting aqueous slurry by a highly multiple-stage further treatment, which includes the removal of ammonia and carbon dioxide, the dissolving of the melamine in a large amount of alkaline water, removal of color with activated carbon, crystallization, filtration, drying, and packaging.

The Montedison process has the significant disadvantage that the number of process stages is very high since the impure product obtained from the reactor requires a thorough purification treatment.

In the Nissan high-pressure process (Ullmann's Encyclopedia of Industrial Chemistry, 5th edition. Vol. A 16, p. 178) advantages have been gained over the Montedison process at least with respect to the offgases, as is evident from the following. Also in the Nissan process, urea melt and hot ammonia are fed into the reactor. The temperature and the pressure are 400° C. and 10 MPa. In the upper section of the reactor, the melamine melt and the gases are separated. The gases are directed into a scrubbing tower, in which they are scrubbed with urea melt. The melamine present in the gas dissolves in the urea melt. At the same time the gases cool to approx. 200° C. The product gas is thus obtained at a pressure of 10 MPa and in anhydrous state, which may be a considerable advantage in terms of its further use. The urea melt to be used as raw material is supplied via the scrubbing tower. There it heats up and water is removed from it. The melamine melt is dissolved in an aqueous ammonia solution. This solution is maintained under ammoniacal pressure at 180° C. for a certain period, during which the impurities are said to be eliminated. Thereafter follows a further treatment with numerous apparatus, including filtration and crystallization.

U.S. Pat. No. 4,565,867 describes a non-catalytic high-pressure process for making melamine in which the quantity of apparatus is quite small, as compared with the Montedison and Nissan processes, as discussed above. After the reaction stage, the offgases are separated from the molten melamine similar to the Nissan process, and the melamine melt is directed to a quencher unit in which it is cooled rapidly by means of, for example, liquid ammonia or water. Crystalline melamine is obtained, which is withdrawn via the bottom of the quencher unit and taken to drying. There are no actual purification stages. However, the purity of the product is only 96-99.5% which does not meet the product specifications that are normally required for most uses of melamine.

Purification of the product by vaporization has been proposed previously. One of the earliest melamine preparation patents (GB Patent 800 722) includes an example in which approx. 9 kg of ammonia per one kilogram of melamine product is fed into a reactor which operates at the temperature of 400° C. and under a pressure of 4-8 MPa. The amount of ammonia in this case is so high that all of the produced melamine is vaporized into the gas phase. The promoting effect of ammonia on melamine vaporization is based on the fact that it reduces the partial pressure of melamine in the gas phase. The disadvantage of the process described in GB Patent 800 722 is that the melamine has to be removed from a very large amount of gas. Furthermore, before the large amount of ammonia can be recycled into the reactor for reuse, carbon dioxide has to be separated from it. Thus, the process is uneconomical owing to the large gas amounts and the related separation operations.

To overcome this problem Nissan has investigated a process, as described in U.S. Pat. No. 3,484,440 that can be managed with a smaller amount of ammonia, in which case the melamine content is many times higher in the offgas. When the pressure and temperature are selected suitably within the proposed limits, all of the melamine can be caused to vaporize into the gas phase. Before vaporization, however, the melamine is allowed to remain in the vaporizer in the form of a liquid melt for a minimum of one hour to convert the impurities formed in the reaction into melamine. From the vaporizer the melamine is directed to a separator, in which it is cooled with water, and will crystallize. The sole example discloses a melamine purity of 99.2%, which is still not competitive in terms of purity.

To overcome the purity problem resulting from the process in U.S. Pat. No. 3,484,440 a similar process has been proposed in WO 95/01345. Although this process also utilizes evaporation of the produced melamine, the reaction product obtained from the pyrolysis of urea comprising, as described above, liquid melamine and offgases consisting essentially of carbon dioxide, ammonia and traces of melamine, is first separated in a liquid gas separator. Only after separation of the gaseous byproducts the liquid melamine melt thus obtained is first vaporized by feeding ammonia into a vaporizer and the resulting gas mixture containing ammonia and melamine is fed into a quencher in which the melamine is crystallized. Thereby a very pure product having a purity of 99.9% is obtained. According to the teaching of WO 95/01345 removal of carbon dioxide from the reaction mixture prior to evaporation of the molten melamine is essential to obtain the high-purity melamine.

According to WO 00/71525 the process described in WO 95/01345 still suffers from the problem that although CO₂ is removed from the melamine melt prior to evaporation still oxygen-containing compounds, in particular ammelide, ammeline, and ammonium isocyanate, are not removed and these compounds still produce carbon dioxide. The thus produced carbon dioxide will react with ammonia in the quench section to produce ammonium carbonate. In the quencher, of course, it is economically preferred to recycle all of the gas stream. But this would accumulate CO₂ resulting in fouling of the filter and the ammonia condensers caused by ammonium carbamate formation. As solution to this problem WO 00/71525, therefore, suggests to evaporate not only reactor offgases from the molten melamine but to strip the molten melamine by addition of ammonia in a stripper to obtain a purified melamine melt prior to the evaporation step.

Consequently, WO 95/01345 as well as WO 00/71525 consider it essential not only to separate the reactor offgases from the molten melamine (WO 95/01345) but also to strip the molten melamine in order to produce a purified liquid melamine stream (WO 00/71525) prior to the evaporation step. Thus, according to both teachings additional vessels to be operated at a high pressure are necessary which is undesirable in terms of investment and operation costs.

An alternative approach trying to improve the low pressure catalytic process for making melamine from urea is disclosed in WO 96/20933. Herein a catalytic low pressure process operating at a pressure range of 1.4 to 2 MPa is disclosed. The reactor effluent vapor stream contains melamine, ammonia and carbon dioxide. This vapor is first quenched by contact with an aqueous mother liquid from a melamine purification unit. Thereafter the obtained melamine-rich solution is stripped in order to remove ammonia and carbon dioxide. The vapor components from the stripping zone are then washed to remove residual melamine with a mother liquid stream from melamine purification. The essentially melamine-free vapor stream comprising ammonia, carbon dioxide and water vapor drawn from the wash zone is then fed to an absorption zone comprising liquid ammonia and aqueous ammonia to absorb carbon dioxide and water and produce a concentrated ammonium carbamate stream that may be fed to the urea production.

Several proposals were made to improve the process described in WO 96/20933 in order to reduce the water content of the exported ammonium carbamate solution obtained in the absorber unit. WO 01/056999 and WO 2/14289 both suggest to use an aqueous ammonium carbamate stream obtained from an ammonium carbamate absorption unit as quench liquid to cool the gaseous effluent from the melamine reactor.

WO 2001/057000 additionally suggests to cool the aqueous ammonium carbamate stream before it is fed to the quencher.

All the above described catalytic low pressure processes have the principal disadvantage of low pressure processes, i.e. large reactor and other process unit volumes resulting in high investment costs. Furthermore, all these processes suffer from the problem of catalyst entrainment which makes it necessary that after the quenching step the melamine has to be dissolved in water, followed by removal of the entrained catalyst by suitable means, i.e. filtration, and subsequent recrystallization. Thus, recovery of pure solid melamine requires additional complicated process steps. Furthermore, in the catalytic low-pressure processes quenching of the gaseous effluent from the reactor is slow, resulting in the formation of undesired by-product which again makes additional purification steps necessary.

Thus, it is the object of the present invention to overcome the above described disadvantages of the so far known melamine processes. It is especially the object to provide a melamine process wherein melamine of high purity can be obtained in an economic manner by using a minimum number of high-pressure process units and a minimum number of purification steps at a high throughput.

### SUMMARY OF THE INVENTION

These objects have been surprisingly attained by a non-catalytic process for the preparation of melamine comprising:
a) reacting molten urea in a reaction section at a pressure of 4.5 to 15 MPa to produce a reaction mixture containing molten melamine and reaction off-gases;
b) evaporating the molten melamine in said reaction mixture in an evaporization section to produce a gaseous mixture comprising the vaporized melamine and the reaction off-gases at a pressure of 4.5 to 15 MPa;
c) quenching the gaseous mixture obtained from step b) by contact with an aqueous ammonium carbamate solution; and
d) isolating melamine.

The process of the present invention has several advantages compared to the above discussed teaching of the prior art.

The process of the present invention being a non-catalytic process operated at a pressure of 4.5-15 MPa utilizes the improved economics of high-pressure processes, especially small reactor volumes. But contrary to the teachings of WO 95/01345 and WO 00/71525, neither a gas/liquid evaporation step, nor a stripping step is necessary between reaction and evaporation of the molten melamine in order to obtain highly purified melamine. Thus, the number of high-pressure process units in the process of the present invention is reduced which results in a further improvement in terms of investment and operation costs without compromising purity of the produced melamine.

Compared to the above discussed catalytic low-pressure processes no entrained catalyst has to be removed from the melamine product simplifying the recovery of the melamine product.

An additional advantage of the process of the present invention compared to the catalytic low-pressure process is the reduced quench time which, again, suppresses the formation of undesired impurities during the quench step.

According to a preferred embodiment of the present invention the gaseous phase comprising water, ammonia and carbon dioxide formed in the quench step c) is at least partially condensed, optionally in presence of additional water in a condensation/absorption step d) to form a concentrated aqueous ammonium carbamate solution. Preferably part of the obtained concentrated aqueous carbamate solution is recycled to the quench step and part of the concentrated aqueous ammonium carbamate solution is transferred to another plant, e.g. a urea plant.

A particular advantage of this preferred embodiment of the present invention is that it allows a broader flexibility for adjusting the process conditions, especially pressure conditions, in the quench step c) and the condensation/absorption step d), to find an optimum balance of quench time and concentration of the aqueous ammonium carbamate stream. Thereby a highly concentrated ammonium carbamate stream to be transferred to a urea plant can be obtained without compromising a short quench time.

Thus, the process of the present invention can be advantageously combined with a urea preparation process.

Consequently, the present invention also relates to a combined melamine/urea process.

### DETAILED DESCRIPTION OF THE PRESENT INVENTION

According to the process of the present invention melamine is produced using urea as raw material. The urea is fed as a melt into a reaction section and is reacted at a pressure of 4.5 to 15 MPa, preferably 5 to 8 MPa at elevated temperature to form melamine and the by-products ammonia and carbon dioxide in accordance with the above mentioned reaction equation. The reaction conditions in the reaction zone are selected in order to obtain melamine in the liquid state. The reaction temperature is preferably 360 to 440° C., more preferred 400° C. to 430° C., even more preferred 401° C. to 419° C.

The thus obtained reaction mixture comprising molten melamine and the gaseous reaction by-products carbon dioxide and ammonia, is then subjected to a vaporization step whereby, without separating the molten melamine from the gaseous reaction by-products, the melamine is vaporized in order to form a gaseous mixture comprising the vaporized melamine and the reaction offgases.

Vaporization can be achieved by any means known to the person skilled in the art, like increase of temperature, reduction of pressure, or both. Preferably the pressure is kept approximately constant, and vaporization is achieved by feeding ammonia into the vaporization section in order to reduce the partial pressure of melamine, thereby vaporizing the melamine. In accordance with this preferred embodiment of the present invention vaporization of the melamine can be achieved by feeding ammonia in an amount of 0.5 to 3 kg ammonia/kg melamine to the vaporization section, preferably 1.05 to 1.9 kg ammonia/kg melamine at an evaporator temperature between 401° C. and 419° C. Thus, only limited amounts of ammonia are needed, resulting in smaller volumes of ammonia to be processed in process steps subsequent to the vaporization step of the present invention. Thus, smaller process units are sufficient to handle the ammonia streams generated in the process of the present invention, thereby further improving the economics of the present process. Preferably the melamine content in the gaseous phase from the evaporator is lower than the saturation pressure of melamine at the prevailing process conditions.

In general, the reaction step a) and the evaporation step b) according to the present invention can be conducted in different vessels, but it is preferred to conduct these process steps in different sections of a vessel. Preferably the reactor/evaporator contains a draught tube for improving the contact between the liquid melamine and the ammonia gas. The ammonia may be split to the different sections, irrespective whether they are part of the same or different vessels.

The gaseous mixture obtained from the vaporization step comprising melamine, carbon dioxide and ammonia is directed to a cooling unit wherein the gas mixture is quenched by direct contact with an aqueous ammonium carbamate solution. The pressure in the quenching step c) is preferably at least 0.5 MPa lower than in the evaporation step b). In order to achieve an even faster quenching it is preferred that the pressure in the quenching step is less than 60%, even more preferred less than 75% of the pressure in the evaporation step b). As will be discussed in more detail below with respect to the condensation/absorption step d) in accordance with a preferred embodiment of the present invention, the pressure in the quenching step c) is preferably at least 1.6 MPa, more preferred at least 1.9 MPa, and most preferred at least 2.2 MPa.

In the cooling unit upon quenching the gaseous reaction product a liquid aqueous phase comprising melamine and a gaseous phase comprising water, ammonia and carbon dioxide, is formed.

After quenching, the liquid phase is separated from the gaseous phase for further processing.

The liquid aqueous phase can be either an aqueous melamine solution or an aqueous melamine slurry. An aqueous melamine slurry is more preferred since the melamine can be directly isolated by conventional liquid/solid separation techniques, like filtration. No further purification steps for the melamine are necessary in order to obtain highly pure melamine of more than 99.5 or even more than 99.9% purity.

The gaseous phase comprising water, ammonia and carbon dioxide separated from the quenching step is preferably directed to a condensation/absorption step d) where the gaseous phase is at least partially condensed, optionally in presence of additional water to form a concentrated aqueous ammonium carbamate solution and a gas comprising ammonia. For example, water, optionally together with carbon dioxide and ammonia, may be obtained from stripping the aqueous ammonium carbamate solution in the work-up section in order to isolate solid melamine and then recycled to the condensation/absorption step d).

The pressure in the condensation/absorption step d) is in the same order as in the quenching step c). Higher pressures in the absorption condensation step d) are preferred in order to produce higher concentrated ammonium carbamate solutions that can be directly used without any intermediate concentration steps in a urea plant. Thus it is preferred to operate the quenching step c) and thus the condensation and absorption step d) at a pressure of at least 1.6 MPa, preferably at least 1.9 MPa, and more preferred at least 2.2 MPa, as already mentioned above.

Furthermore, it is an advantage of the process of the present invention that the pressure drop between the evaporation step and the quenching step can be adjusted to find an optimum balance between fast quenching (achieved by high pressure differences between the evaporation step and the quenching step) and high concentration of the ammonium carbamate solution obtained in the absorption condensation step (achieved by high pressure in the condensation absorption step). Thus, the process according to the present invention provides a highly concentrated ammonium carbamate solution that can be directly introduced into a urea plant without a further concentration step. Furthermore, part of the obtained highly concentrated ammonium carbamate solution is preferably recycled to the quenching step. The concentrated carbamate solution obtained in the condensation/absorption step contains less than 50 wt.-% water, preferably less than 30 wt.-% water

The gaseous effluent from the condensation/absorption step d) consists essentially of ammonia and can be, after optional separation or purification steps and after repressurization, recycled to the reactor/evaporation unit. The gaseous ammonia from the condensation/absorption section may be condensed partially and used as a reflux to increase the purity of the gaseous ammonia. Virgin liquid ammonia may also be used as an absorption liquid for purification of ammonia gas.

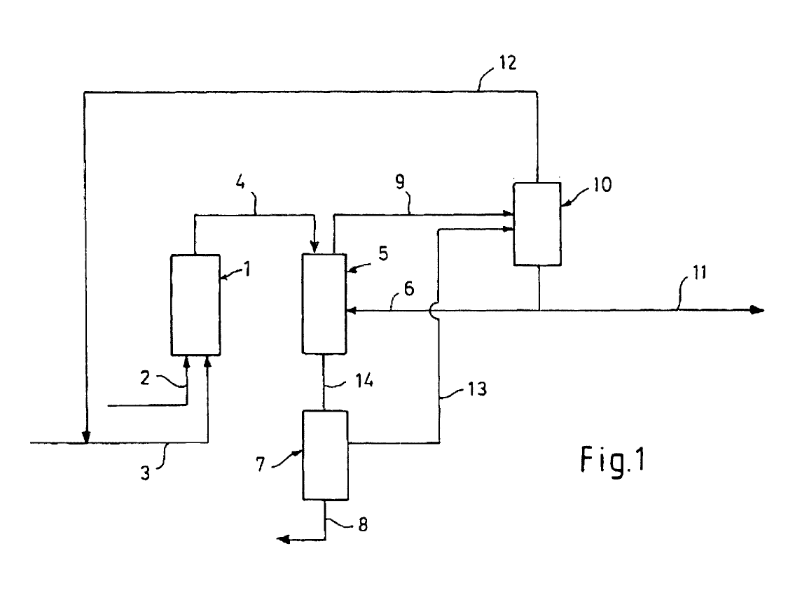

The present invention will now be discussed in more detail with reference to a preferred embodiment that is shown in a schematic representation in FIG. 1. Molten urea and hot ammonia are fed via line 2 and line 3, respectively, to a reactor/evaporator 1 which is depicted in FIG. 1 as a single process unit comprising a reaction and a vaporization section. The gaseous mixture obtained from the vaporization step comprising melamine, carbon dioxide and ammonia, is fed via line 4 to a cooling unit 5 where the gaseous mixture is quenched by contact with an aqueous carbamate solution that is fed into the cooling unit 5 via line 6. The cooling unit 5 contains also a gas/liquid separator allowing separation of a gaseous effluent comprising water vapor, carbon dioxide and ammonia, and an aqueous phase comprising melamine, preferably in form of an aqueous melamine slurry.

The aqueous melamine slurry is fed via line 14 to a process unit 7 where solid melamine is separated from the aqueous phase. The aqueous phase can be processed as known by a person skilled in the art. For example, the aqueous phase may be stripped and the resultant gaseous phase may be fed via line 13 to the condensation/absorption unit 10. Solid melamine is removed via line 8 for drying and further utilization. The gaseous effluent from the quenching step is directed via line 9 to the condensation/absorption unit 10 where it is condensed/absorbed to form a highly concentrated ammonium carbamate solution that is partially fed via line 6 to the cooling unit 5 and the remainder is directed via line 11 to a urea plant (not shown). The non-condensed gaseous phase predominantly consisting of ammonia after optional separation step (not shown) and repressurization is recycled to the reactor/vaporizer 1 via line 12. The gaseous ammonia can be condensed before repressurization followed by evaporation at higher pressure. This embodiment is preferred at a high pressure difference between the reaction/evaporation section and the quench section.

The invention will be explained in more detail with reference to the following Examples and comparative experiments.

### Examples 1 and 2

Liquid melamine was produced from urea melt (1.4 t/h, 140° C.) at 5.5 MPa in a combined liquid-phase reactor/evaporator, which was heated with molten salt. The liquid melamine was evaporated at 419° C. by introducing 1.7 t/h ammonia of 330° C. The gas from the reactor/evaporator (containing mainly ammonia, CO₂ and melamine vapor) was quenched rapidly in a cooling tower at a pressure Pc with an aqueous carbamate solution originating from an absorption/condensation unit. The quench time is defined as the time needed to cool the melamine containing gas to 250° C. A melamine slurry in aqueous carbamate solution and quench offgas were produced. The quench offgas was sent to an absorption/condensation unit operating at almost the same pressure as the cooling tower. In the absorption zone water and CO₂ were removed from the quench offgas by partial condensation and by washing with liquid ammonia, producing an aqueous carbamate solution (CS) as a bottom stream and ammonia gas as a top stream. Part of the aqueous carbamate solution was returned to the cooling tower and used as a cooling agent. Water was added to his carbamate solution before returning to the cooling tower to balance the water export.

### Comparative Experiments A and B

Gaseous melamine was produced in gas fluidized-bed reactor at 2 MPa and 419° C. by introducing 1.4 t/h urea melt (140° C.) and 1.7 t/h ammonia of 330° C. to the reactor. The gas from the reactor (containing mainly ammonia, CO₂ and melamine vapor) was quenched rapidly in a cooling tower at a pressure Pc with an aqueous carbamate solution originating from an absorption/condensation unit. The quench time is defined as the time needed to cool the melamine containing gas to 250° C. A melamine slurry in aqueous carbamate solution and quench offgas were produced. The quench offgas was sent to an absorption/condensation unit operating at almost the same pressure as the cooling tower. In the absorption zone water and CO₂ were removed from the quench offgas by partial condensation and washing with liquid ammonia producing an aqueous carbamate solution (CS) as a bottom stream and ammonia gas as a top stream. Part of the aqueous carbamate solution was returned to the cooling tower and used as a cooling agent. Water was added to this carbamate solution before returning to the cooling tower to balance the water export.

TABLE

| | Example 1 | Example 2 | Exp. A | Exp. B |
|---|---|---|---|---|
| Reactor pressure Pr (MPa) | 5.5 | 5.5 | 2.0 | 2.0 |
| Quench pressure Pc (MPa) | 4.1 | 1.95 | 1.95 | 1.49 |
| Ratio Pc/Pr | 0.745 | 0.355 | 0.975 | 0.745 |
| Quench time(s) | 1.8 | 1.5 | 4.2 | 2.6 |

Composition of CS

| | | | | |
|---|---|---|---|---|
| NH₃ (w %) | 45 | 41 | 41 | 38 |
| CO₂ (w %) | 43 | 39 | 39 | 36 |
| H₂O (w %) | 12 | 20 | 20 | 26 |

Comparative Experiments A and B reflect the teaching of WO 01/056999. As can be seen from the experimental data the process of the present invention results in a reduced quench time and in its preferred embodiments at the same time in a highly concentrated ammonium carbamate solution for export to a urea plant.

1. A non-catalytic process for the preparation of melamine comprising:
a) reacting molten urea in a reaction section at a pressure of 4.5 to 15 MPa to produce a reaction mixture containing molten melamine and reaction off-gases;
b) vaporizing the molten melamine in said reaction mixture in a vaporization section to produce a gaseous mixture comprising the vaporized melamine and the reaction off-gases at a pressure of 4.5 to 15 MPa;
c) quenching the gaseous mixture obtained from step b) by contact with an aqueous ammonium carbamate solution; and
d) isolating melamine.

2. The process of claim 1, wherein the molten urea is reacted at a pressure of 5 to 8 MPa.

3. The process of claim 1, wherein the molten urea is reacted at a temperature of 360° C. to 440° C., preferably 400° C. to 430° C.

4. The process of claim 1, wherein the molten melamine is vaporized be feeding ammonia, preferably in an amount of 0.5 to 3 kg ammonia/kg urea to the vaporization section.

5. The process of claim, wherein reacting the molten urea and vaporizing the molten melamine is conducted in different sections of the same vessel or in separate vessels.

6. The process of claim 1, wherein the pressure in the quenching step c) is at least 0.5 MPa lower than in the vaporization step b), preferably the pressure in the quenching step c) is less than 60%, more preferred less than 75% of the pressure in the vaporization step b).

7. The process of claim 1, wherein the pressure in the quenching step c) is at least 1.6 MPa, preferably at least 1.9 MPa and more preferred at least 2.2 Mpa.

8. The process of claim, wherein in the quenching step c) a liquid aqueous phase comprising melamine and a gaseous phase comprising water, ammonia and carbon dioxide is formed.

9. The process of claim 8, wherein the liquid aqueous phase is an aqueous melamine solution or aqueous melamine slurry.

10. The process of claim 8, wherein the gaseous phase obtained in the quenching step c) is at least partially condensed optionally in presence of additional water in a condensation/absorption step d) to form a concentrated aqueous ammonium carbamate solution and an gaseous effluent comprising ammonia.

11. The process of claim 10, wherein the pressure in the condensation/absorption step d) is in the same order as in the quenching step.

12. The process of claim 10, wherein at least part of the concentrated aqueous ammonium carbamate solution is recycled to the quenching step c).

13. The process of claim 10, wherein at least part of the concentrated aqueous ammonium carbamate solution is transferred to an urea plant as at least part of the feed stock.

14. The process of claim 10, wherein the gaseous effluent comprising ammonia after optional purification is recycled to the reaction (a) and/or evaporation step (b).

15. A combined process for the preparation of urea and melamine, wherein melamine is prepared in accordance to the method of claim 13, urea is prepared utilizing said concentrated aqueous carbamate solution as part of the feed stock and urea obtained from said preparation step is supplied in molten form to the reacting step a).
